(19) United States
(12) Patent Application Publication
(10) Pub. No.: US 2018/0344161 A1
(43) Pub. Date: Dec. 6, 2018

(54) MEDICAL INSTRUMENT FOR ANALYSIS OF WHITE MATTER BRAIN LESIONS

(71) Applicant: KONINKLIJKE PHILIPS N.V., EINDHOVEN (NL)

(72) Inventors: CARSTEN MEYER, EINDHOVEN (NL); FABIAN WENZEL, EINDHOVEN (NL); MARTIN BERGTHOLDT, EINDHOVEN (NL); THOMAS HEIKO STEHLE, EINDHOVEN (NL); LYUBOMIR GEORGIEV ZAGORCHEV, EINDHOVEN (NL)

(21) Appl. No.: 15/774,771

(22) PCT Filed: Nov. 11, 2016
(86) PCT No.: PCT/EP2016/077507

§ 371 Date: May 9, 2018

Related U.S. Application Data
(60) Provisional application No. 62/254,236, filed on Nov. 12, 2015.

(51) Int. Cl. A61B 5/00; A61B 5/055; G06T 7/11; G06T 7/00

(52) U.S. Cl. CPC A61B 5/0037; A61B 5/0035; A61B 5/0042; A61B 5/055; A61B 2090/364; A61B 5/7282; A61B 5/7485; G06T 7/11; G06T 7/0016; A61B 5/4064

(57) ABSTRACT

The present invention relates to a medical instrument for automatically detecting affected regions in an examination area of a subject comprising: a memory containing machine executable instructions; and a processor for controlling the medical instrument, wherein execution of the machine executable instructions causes the processor to control the instrument to: obtain a first anatomical image of the examination area and a first image of fibers of the examination area, wherein a first parameter and a second parameter describe characteristics of the first anatomical image and the first image of fibers respectively; segment the first anatomical image into a plurality of segments indicating respective tissues and/or structures in the examination area; identify first lesions in the segmented first anatomical image; use values of the first and/or second parameters for determining seed points in the identified first lesions for a tracking algorithm for tracking first fibers in the first image of fibers.

### TECHNICAL FIELD OF THE INVENTION

The invention relates to magnetic resonance imaging systems, in particular to a method for automatically identifying lesions in an examination area.

### BACKGROUND OF THE INVENTION

White matter lesions are widely observed especially in elder patients and are associated with cognitive and psychomotoric deficiencies. The cognitive impact of white matter change may depends on its location, where e.g. periventricular white matter lesions may affect cognition more than deep white matter lesions. Therefore, the assessment of the severity, location and progression of white matter lesions becomes important. Also, the regional assessment and statistical analysis of white matter lesions as well as the visualization of white matter tracts affected by the white matter lesions and the respective target area on the cortex is important for the patients' diagnosis and prognosis. Currently, however, such an analysis requires substantial interactions to e.g. configure a fiber tracking algorithm.

M. Caligiuri et al., Neuroinformatics 13:261-276 (2015), reviews the state-of-the-art in automatic detection of white matter hyperintensities or lesions in healthy aging and pathology using magnetic resonance imaging.

### SUMMARY OF THE INVENTION

Various embodiments provide a medical instrument, a computer program product and a method as described by the subject matter of the independent claims. Advantageous embodiments are described in the dependent claims. Embodiments of the present invention can be freely combined with each other if they are not mutually exclusive.

Various embodiments provide a medical instrument for automatically detecting affected regions in an examination area of a subject. For example, the medical instrument may detect affected grey matter regions on the cortex surface. The medical instrument comprises: a memory containing machine executable instructions; and a processor for controlling the medical instrument, wherein execution of the machine executable instructions causes the processor to control the instrument to:

a) obtain a first anatomical image of the examination area and a first image of fibers of the examination area, wherein a first parameter and a second parameter describe characteristics of the first anatomical image and the first image of fibers respectively;
b) segment the first anatomical image into a plurality of segments indicating respective tissues and/or structures in the examination area;
c) identify first lesions in the segmented first anatomical image;
d) use values of the first and/or second parameters for determining seed points in the identified first lesions for a tracking algorithm for tracking first fibers in the first image of fibers. For example, step d) may in particular comprise determining values of the first and second parameters.

For example, the seed points may first be placed in the identified first lesions using values of the first parameter e.g. using the methods described herein for determining seed points such as the center of gravity method. For example, each seed point may be placed in a respective first lesion. Once seed points are placed, values of the second parameter may be matched with (or verified for) each placed seed point, and then it is decided based on the verification whether to use or not use the seed point for the tracking of fibers.

The term “anatomical image” as used herein refers to a medical image obtained with methods with resolved anatomic features, such as by X-ray, computer tomography (CT), magnetic resonance imaging (MRI) and ultrasound (US). The tracked first fibers start or pass through first lesions to affected first cortical areas. The first anatomical image and the first image of fibers are registered.

The first anatomical image and the first image of fibers may automatically be scanned at the same time or concurrently in order to use the characteristics of the first anatomical image and the first image of fibers such that the seed point is first positioned or placed in a given first lesion of the identified first lesions and a decision based on the comparison (or evaluation of the second parameter) is made to use or not use the placed seed point as starting point for the tracking algorithm. The comparison may comprise for example placing the seed point and comparing the values of the second parameter for the seed point with a threshold.

The first image of fibers may for example be obtained using the diffusion tensor imaging, diffusion weighted imaging or diffusion tensor tractography technic.

The term “lesion” as used herein refers to an abnormality in the tissue of an organism such as a body of a patient, usually caused by disease or trauma. Lesions may occur in the body that consists of soft tissue (fat tissue, muscles, skin, nerves, blood vessels, spinal disks, etc.) or osseous matter (spine, skull, hip, ribs, etc.) or organs (lungs, prostate, thyroid, kidney, pancreas, liver, breast, uterus, etc.), such as in the mouth, skin, and the brain, or anywhere where a tumor may occur. The term “lesion” may also refer to abnormalities caused by cancerous diseases, like oropharyngeal, adrenal, testicular, cervical, spinal or ovarian tumors as well as tumors or carcinomas located at the skin (melanoma) and in the lungs, prostate, thyroid, kidney, pancreas, liver, breast, uterus, etc.

The term “fiber” as used herein refers to a fiber path through a specimen that can be followed from voxel to voxel of an image of fibers e.g. the first image of fibers. The fiber may for example comprise a nerve fiber or a muscle fiber or a bundle of such fibers. The term “fiber” can mean a single fiber or a bundle of fibers. Fiber tracking (e.g., tractography) may be based on a variety of tracking algorithms. For example, fiber trajectories can be based on principal axis directions tracked from voxel to voxel in three dimensions based on the diffusion tensor in a local neighborhood, starting at the seed points. Fiber direction is mapped by following principal axis directions and changes at voxel edges as principal axis directions change. A variety of tracking methods can be used as well, including sub-voxel based tracking methods, high definition fiber tracking (HDFT) method, probabilistic methods, and methods associated with selection of suitable seed voxels from which fiber tracking is to start.

For example the examination area may comprise the brain of a patient. For example, the lesion may comprise a white matter lesion.

In one example, the present method may be applied when surgeons are trying to protect tracts that affect movement, or speech. In such cases it is important to identify and visualize specific tracts (related to the pre-surgical planning) in order to preserve them during the procedure.

The above features may have the advantage of enabling an automatic fiber (e.g. white matter fiber) tracking without manual intervention. This may avoid the tedious procedure of manual interventions in particular for a case of a substantial number of lesions (e.g. white matter lesions). In particular, it may appear to be impossible to manually handle all white matter lesions in an anatomical region of interest.

Another advantage may be that the present method may speed up the process of tracking fibers compared to the manual method, and may provide accurate and reliable results.

According to one embodiment, the first parameter comprises at least one of size, voxel intensity, number, fractional volume of the identified lesions. For example, each first lesion of the identified first lesions may cover a respective number of voxels in the first anatomical image, wherein each voxel of the number of voxels has a voxel intensity. The second parameter comprises at least one of the direction of diffusion and the magnitude of the diffusion in the first image of fibers. The first image of fibers may comprise a diffusion weighted image.

The seed points are determined not only from the identified first lesions but also using the first image of fibers. For example, a seed point may first be placed in a given identified first lesion (e.g. a voxel having the highest or lowest intensity among voxels representing the given identified first lesion) and before using the seed point for the tracking, values of the second parameter may be checked. For example, based on the diffusion directions in the first image fibers it may be decided whether the seed point matches at least one of those diffusion directions. In this case, only if there is a match, the seed point is used for tracking. This may have the technical advantage of automatically, in an accurate manner, detecting affected regions (e.g. affected grey matter regions) in an examination area.

Various embodiments provide for a medical instrument comprising: a memory containing machine executable instructions; and a processor for controlling the medical instrument, wherein execution of the machine executable instructions causes the processor to control the instrument to:

a) obtain a first anatomical image of an examination area of a subject and a first image of fibers of the examination area;
b) segment the first anatomical image into a plurality of segments indicating respective tissues and/or structures in the examination area;
c) identify first lesions in the segmented first anatomical image;
d) use the identified first lesions as seed points for a tracking algorithm for tracking first fibers in the first image of fibers.

According to one embodiment, execution of the machine executable instructions further causes the processor to control the instrument to:

e) obtain a second anatomical image of the examination area and a second image of fibers of the examination area;
f) segment the second anatomical image into a plurality of segments indicating respective tissues and/or structures in the examination area;
g) identify second lesions in the segmented second MR image;
h) use the identified second lesions as seed points for the tracking algorithm for tracking second fibers in the second image of fibers;
i) compare at least the first and second lesions;
j) provide data indicative of the difference between imaged first and second lesions and repeat steps e)-j) until a predefined convergence criterion is fulfilled.

For example, step i) may further comprise comparing the first tracked fibers and second tracked fibers. In another example, step i) may further comprise comparing affected first and second cortical areas in the examination area in case the examination area comprises the brain.

For example, step j) may further comprise providing data indicative of the difference between first and second lesions, between affected first and second fibers and/or between affected first and second cortical areas. If, for example, a first lesion of the first lesions grows during the time interval between the image acquisitions of the first and second anatomical images, and this growth occurs in the direction of the affected first fibers, the effect of the lesion growth on the affected first cortical area may be small. In contrast, if the lesion growth occurs mainly in the direction perpendicular to the affected first fibers, the lesion growth may affect additional fibers and thus also the affected first cortical area may grow.

For example, the repeating of steps e)-j) may automatically be performed on a periodic basis e.g. every year etc. In another example, the repeating of steps e)-j) may be triggered by a user of the medical instrument. For example, steps e)-j) may be performed for two sets of images in order to perform a longitudinal analysis. The first set of images comprises the first anatomical image and the first image of fibers. The second set of images comprises the second anatomical image and the second image of fibers. The first set of images is obtained or acquired at a first point in time and the second set of images is obtained or acquired at a second point in time. The first and second set of images may be chosen or selected from a pool of set of images. For example, the pool of the set of images may comprise more than two sets of images. The selection of the two sets of images may be random or based on user defined criteria. The two sets of images may be registered before performing the longitudinal analysis.

For each repetition or iteration, step e) comprises obtaining a current anatomical image and a current image of fibers of the examination area. For example, the two images that are used in step e) may both be created, reconstructed or generated at a predefined maximum time interval before the time at which the execution of step e) is performed.

The repeating of steps e)-j) may be performed for the same or different patients, wherein the two images used in step e) may be associated with the respective patient in case of different patients. The two images in each iteration are performed for the same examination area e.g. the brain. Repeating steps e)-j) for different patients may be useful for test purposes such as comparing the amount and/or progression of lesions between two patients.

The provision of data indicative of the difference between imaged first and second lesions may comprise displaying on a graphical user interface on a display device of the medical instrument data indicative of the difference. The difference may be quantified for example by a relative difference and/or absolute difference between the imaged first and second lesions. The difference between the imaged first and second lesions refers to the difference between values of a parameter that describes characteristics of the first and second lesions. For example, the parameter may comprise the volume of a lesion, the total volume of the identified lesions, the number of identified lesions and/or a ratio of a white matter lesion volume to cortical area (e.g. ratio of the first lesion's volume to the first cortical area and/or ratio of the second lesion's volume to the second cortical area), where a value of the ratio higher than a predetermined threshold indicates lesion growth along fibers and a value of the ratio smaller than the predetermined threshold may indicate region growth across fibers. For example, in addition to the displayed difference a region-wise profile that represents the characteristics of the lesions (e.g. in a region-of-interest) such as size, number, fractional volume etc. may be generated and displayed on the graphical user interface. The value of the parameter may for example in case of the brain be obtained by analyzing the identified (first and second) lesions with respect to its extension relative to the orientation of fiber bundles passing through the lesion to the cortical region of the brain. The affected cortical surfaces or areas may also be displayed on the graphical user interface. The values of the parameter may be displayed on the graphical user interface. In particular, this embodiment may provide an efficient method for determining the progression of the identified lesions over time with respect to an affected cortical area e.g. for the same patient.

Another advantage may reside in the fact that the present method may enable an automatic longitudinal analysis which may speed up the whole process of longitudinal analysis compared to conventional “ad-hoc” methods.

According to one embodiment, the convergence criterion comprises at least one of: the difference between the imaged first and second lesions is below a predefined threshold; receiving a stopping signal upon performing step j); the number of second lesions is equal to the number of the first lesions. For example, the stopping signal may be triggered by the user of the medical instrument. The user may select a user interface element in the graphical user interface that triggers the stopping signal. This embodiment may further speed up the longitudinal analysis process compared to a case where the stopping is randomly triggered which may induce the need of additional attempts or repetitions if it turns out that the stopping was premature. In another example, the convergence criterion may be predefined before performing the iterations. For example, acquisition of imaging data at various time points, may be performed normally as defined by a physician at a first time point (baseline, t0) and then a second time point (half a year or a year later), and probably a third time point (another half a year or year later). In this case, the number of repetitions of the image acquisition may be limited to the 1 or 2, as predefined by the physician or the user of the medical instrument.

According to one embodiment, execution of the machine executable instructions causes the processor to control the instrument to perform the tracking in a region of interest of the first anatomical image. This may speed up the tracking process and may save processing resources that would otherwise be required to perform the tracking in the whole first anatomical image.

For example, the tracking may be iteratively performed on multiple regions of interests. The multiple regions of interests may be chosen or selected based on the anatomical structure of the first anatomical image or based on other criteria e.g. user defined criteria.

According to one embodiment, the region of interest is user-defined or automatically selected. The automatic selection may further speed up the tracking process. The user defined region of interest may save processing resources that would otherwise be required for multiple (automatic) attempts to define the right region of interest.

According to one embodiment, the first anatomical image comprises a magnetic resonance, MR, image and the first image of fibers comprises a diffusion weighted image.

According to one embodiment, the medical instrument further comprises a magnetic resonance imaging, MRI, system for acquiring magnetic resonance data from the subject, wherein the magnetic resonance imaging system comprises a main magnet for generating a BO magnetic field within an imaging zone and the memory and the processor, wherein execution of the machine executable instructions further causes the processor to control the MRI system to acquire the MR image and the diffusion-weighted image in a same or different scans.

These embodiments may have the advantage of seamlessly integrating the present method in existing MRI systems.

According to one embodiment, execution of the machine executable instructions further causes the processor to acquire the MR image and the diffusion-weighted image in different scans and to register the MR image and the diffusion-weighted image before performing steps a)-d). This may provide a reliable and an accurate identification and tracking of fibers.

According to one embodiment, execution of the machine executable instructions further causes the processor to calculate a center of gravity of each (segmented) lesion of the lesions and use the center of gravities as the seed points. This may further increase the fiber tracking accuracy of the present method.

According to one embodiment, execution of the machine executable instructions further causes the processor to automatically execute steps a)-d).

According to one embodiment, the provided data comprises characteristics of the (first and second) lesions such as size, number, fractional volume of the first and second lesions.

According to one embodiment, the first lesions comprises white matter lesions, and the examination area comprises a brain.

Various embodiments provide for a computer program product for automatically detecting affected regions in an examination area of a subject, the computer program product comprising a computer readable storage medium having program instructions embodied therewith, the program instructions executable by a processor to

a) obtain a first anatomical image of the examination area and a first image of fibers of the examination area, wherein a first parameter and a second parameter describe characteristics of the first anatomical image and the first image of fibers respectively;
b) segment the first anatomical image into a plurality of segments indicating respective tissues and/or structures in the examination area;
c) identify first lesions in the segmented first anatomical image;
d) use values of the first and/or second parameters for determining seed points in the identified first lesions for a tracking algorithm for tracking first fibers in the first image of fibers. The seed points may be used by the tracking algorithm for tracking the first fibers in the first image of fibers.

Various embodiments provide for a method comprising:

a) obtaining a first anatomical image of an examination area of a subject and a first image of fibers of the examination area;
b) segmenting the first anatomical image into a plurality of segments indicating respective tissues and/or structures in the examination area;
c) identifying first lesions in the segmented first anatomical image;
d) using values of the first and/or second parameters for determining seed points in the identified first lesions for a tracking algorithm for tracking first fibers in the first image of fibers using the seed points.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example data may be retrieved over a modem, over the internet, or over a local area network. Computer executable code embodied on a computer readable medium may be transmitted using any appropriate medium, including but not limited to wireless, wireline, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a processor. ‘Computer storage’ or ‘storage’ is a further example of a computer-readable storage medium. Computer storage is any non-volatile computer-readable storage medium. In some embodiments computer storage may also be computer memory or vice versa.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer indicate the effects of the operator's control or manipulation. The display of data or information on a display or a graphical user interface is an example of providing information to an operator. The display may for example comprise a touch sensitive display device.

A ‘hardware interface’ as used herein encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A ‘processor’ as used herein encompasses an electronic component which is able to execute a program or machine executable instruction. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. Many programs have their instructions performed by multiple processors that may be within the same computing device or which may even be distributed across multiple computing devices.

Magnetic resonance image data is defined herein as being the recorded measurements of radio frequency signals emitted by the subject's/object's atomic spins by the antenna of a Magnetic resonance apparatus during a magnetic resonance imaging scan. A Magnetic Resonance Imaging (MRI) image is defined herein as being the reconstructed two or three dimensional visualization of anatomic data contained within the magnetic resonance imaging data. This visualization can be performed using a computer.

It is understood that one or more of the aforementioned embodiments of the invention may be combined as long as the combined embodiments are not mutually exclusive.

### BRIEF DESCRIPTION OF THE DRAWINGS

In the following preferred embodiments of the invention will be described, by way of example only, and with reference to the drawings in which:

FIG. 1 illustrates a magnetic resonance imaging system,

FIG. 2 is a flowchart of a method for automatically identifying lesions in an examination area,

FIG. 3 is a flowchart of an exemplary method for performing a longitudinal analysis,

FIG. 4 depicts a functional block diagram illustrating a medical instrument,

FIG. 5 depicts a schematic visualization of white matter fibers affected by white matter lesions.

### DETAILED DESCRIPTION OF THE EMBODIMENTS

In the following, like numbered elements in the figures are either similar elements or perform an equivalent function. Elements which have been discussed previously will not necessarily be discussed in later figures if the function is equivalent.

Various structures, systems and devices are schematically depicted in the figures for purposes of explanation only and so as to not obscure the present invention with details that are well known to those skilled in the art. Nevertheless, the attached figures are included to describe and explain illustrative examples of the disclosed subject matter.

The present disclosure may concern an advanced approach to the analysis of white matter brain lesions e.g. from Diffusion tensor imaging MRI (DTI-MRI) images. A longitudinal analysis may be performed based on a segmentation in a current DTI-MR image of lesions in the white matter based on a corresponding identified lesion in an earlier DTI-MR image. Furthermore, the progression of the identified lesion is analyzed e.g. with respect to its extension relative to the orientation of fiber bundles passing through the lesion to the cortical region. A further aspect of the present disclosure is to generate a region-wise profile that represents characteristics of the lesions in the region-of-interest, such as size, number, fractional volume etc. This region-wise profile is also updated from time-to-time based on updated images. The present disclosure may be enabled in practice on the basis of a cortical mesh registration that may be faster than a volumetric registration.

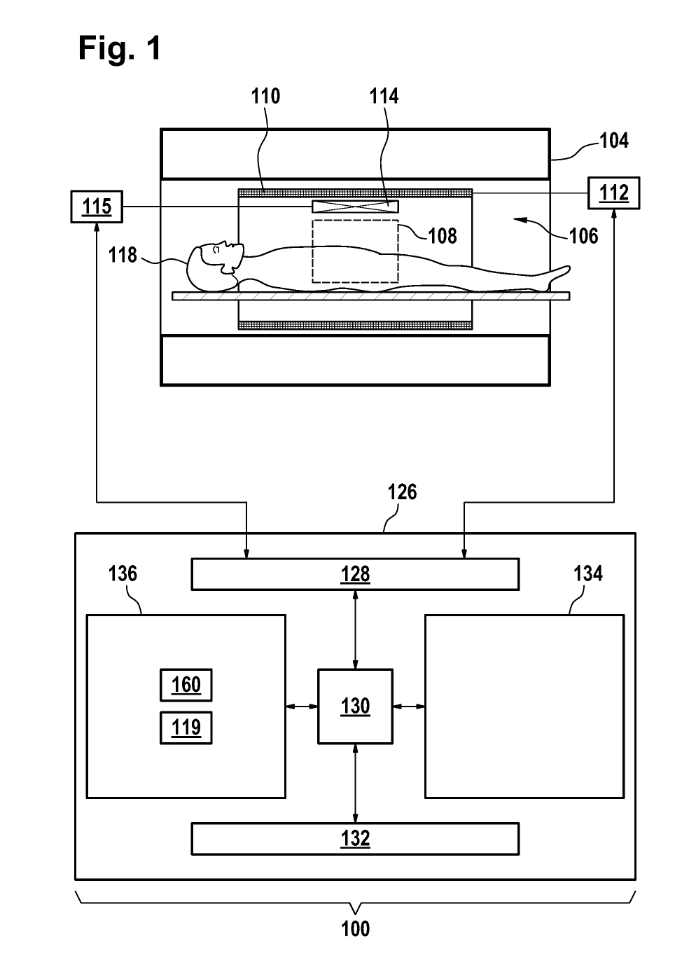

FIG. 1 illustrates a magnetic resonance imaging system 100. The magnetic resonance imaging system 100 comprises a magnet 104. The magnet 104 is a superconducting cylindrical type magnet 100 with a bore 106 in it. The use of different types of magnets is also possible; for instance it is also possible to use both a split cylindrical magnet and a so called open magnet. A split cylindrical magnet is similar to a standard cylindrical magnet, except that the cryostat has been split into two sections to allow access to the iso-plane of the magnet. Such magnets may for instance be used in conjunction with charged particle beam therapy. An open magnet has two magnet sections, one above the other with a space in-between that is large enough to receive a subject 118 to be imaged, the arrangement of the two sections area similar to that of a Helmholtz coil. Open magnets are popular, because the subject is less confined. Inside the cryostat of the cylindrical magnet there is a collection of superconducting coils. Within the bore 106 of the cylindrical magnet 104 there is an imaging zone 108 where the magnetic field is strong and uniform enough to perform magnetic resonance imaging.

Within the bore 106 of the magnet there is also a set of magnetic field gradient coils 110 which is used during acquisition of magnetic resonance data to spatially encode magnetic spins of a target volume within the imaging zone 108 of the magnet 104. The magnetic field gradient coils 110 are connected to a magnetic field gradient coil power supply 112. The magnetic field gradient coils 110 are intended to be representative. Typically magnetic field gradient coils 110 contain three separate sets of coils for the encoding in three orthogonal spatial directions. A magnetic field gradient power supply supplies current to the magnetic field gradient coils. The current supplied to the magnetic field gradient coils 110 is controlled as a function of time and may be ramped or pulsed.

MRI system 100 further comprises an RF coil 114 at the subject 118 and adjacent to the imaging zone 108 for generating RF excitation pulses. The RF coil 114 may include for example a set of surface coils or other specialized RF coils. The RF coil 114 may be used alternately for transmission of RF pulses as well as for reception of magnetic resonance signals e.g., the RF coil 114 may be implemented as a transmit array coil comprising a plurality of RF transmit coils. The RF coil 114 is connected to one or more RF amplifiers 115.

The magnetic field gradient coil power supply 112 and the RF amplifier 115 are connected to a hardware interface 128 of computer system 126. The computer system 126 further comprises a processor 130. The processor 130 is connected to the hardware interface 128, a user interface 132, a computer storage 134, and computer memory 136.

The computer memory 136 is shown as containing a control module 160. The control module 160 contains computer-executable code which enables the processor 130 to control the operation and function of the magnetic resonance imaging system 100. It also enables the basic operations of the magnetic resonance imaging system 100 such as the acquisition of magnetic resonance data and/or diffusion weighted data.

The MRI system 100 may be configured to acquire imaging data from the patient 118 in calibration and/or physical scans.

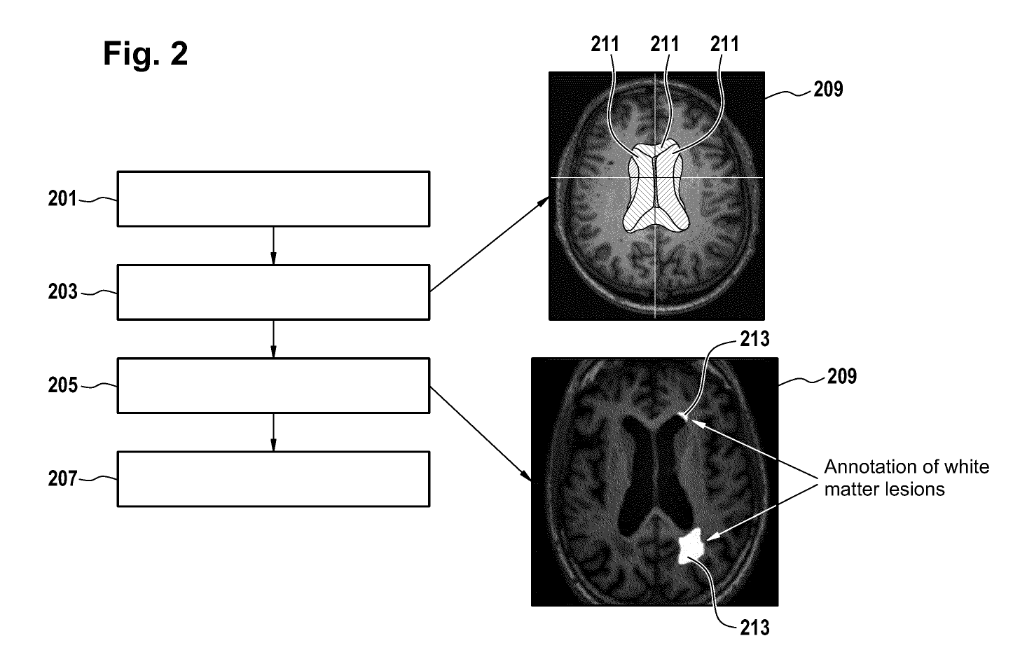

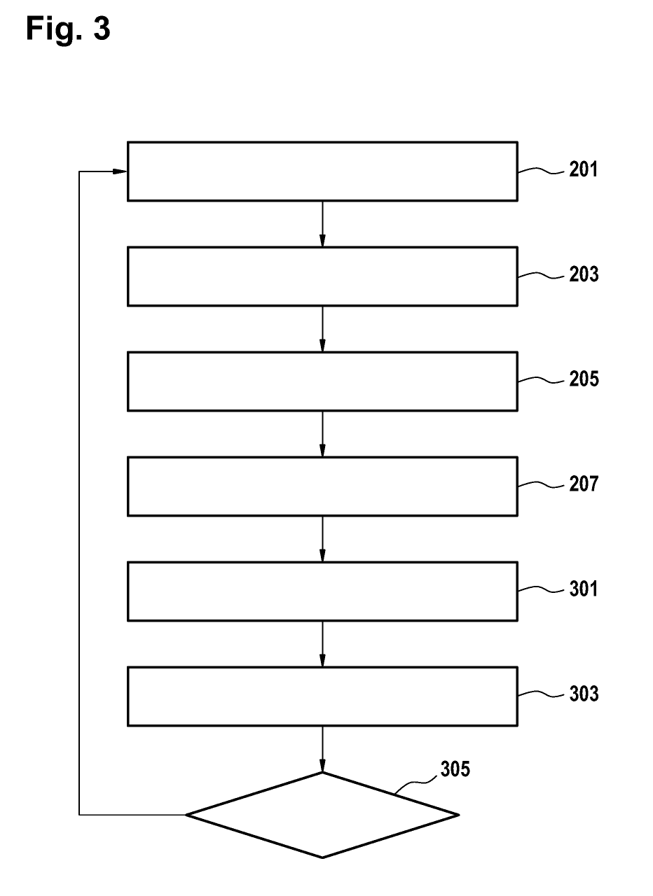

The computer memory 136 is configured to store a lesion detection application 119 comprising instructions that when executed by the processor 130 cause the processor to perform at least part of the method of FIG. 2 and FIG. 3.

FIG. 2 is a flowchart of a method for automatically detecting affected regions in an examination area of a subject e.g. 118.

In step 201, a first anatomical image of the examination area and a first image of fibers of the examination area may be obtained. The first anatomical image may comprise for example a T1 weighted or T2 weighted MR image or a proton density-weighted (PD) or a fluid-attenuated inversion-recovery (FLAIR) MR image. The first image of fibers comprises a diffusion-weighted image or the like.

The obtaining of the first anatomical image and the first image of fibers may comprise receiving the first anatomical image and the first image of fibers from a user. The term “user” as used herein may refer to an entity e.g., an individual, a computer, or an application executing on a computer that inputs or issues requests to process the first anatomical image and the first image of fibers.

The receiving of the first anatomical image and the first image of fibers may be in response to sending a request to the user. In another example, the receiving of the first anatomical image and the first image of fibers may be automatic as the user may periodically or regularly send the received first anatomical image and the first image of fibers.

In another example, the obtaining of the first anatomical image and the first image of fibers may comprise reading from a storage device the first anatomical image and the first image of fibers.

In another example, the obtaining of the first anatomical image and the first image of fibers may comprise controlling the MRI system 100 to acquire MR data and diffusion weighted data of the examination area and to respectively reconstruct therefrom the MR image and the diffusion-weighted image in a same or different scans, wherein the first anatomical image comprise the MR image and the first image of fibers comprises the diffusion-weighted image. In case the MR image and the diffusion weighted image are acquired using different scans the obtaining of step 201 may further comprise controlling the MRI system 100 to register the MR image and the diffusion-weighted image.

In step 203, the first anatomical image 209 may be segmented into a plurality of segments 211 indicating respective tissues and/or structures in the examination area (tissues may be used to indicate where lesions are; structures may be used to indicate where the anatomical location of the lesion is (with respect to organ structures)). In case the examination area comprises the brain, the tissues of the segmented first anatomical image may be at least one of white matter, gray matter, cerebrospinal fluid (CSF), edema and tumor tissue.

The segmenting may comprise dividing up the first anatomical image into a patchwork of regions or segments each of which is homogenous e.g. in term of intensity and/or texture. For example, the segmenting may comprise assigning to each individual element of the first anatomical image a tissue class indicating the tissue to which belongs the individual element. The individual element may comprise a voxel. The tissue class may be assigned to the individual element by for example assigning a value e.g. number, specific to that tissue class. For example, each individual element of the first anatomical image may be classified according to its probability of being a member or part of a particular tissue class. For example, the structure and tissue segmentations may be accomplished by same or different algorithms. The shape-constrained deformable models may for example be used for the segmentation. In another example, the segmentation may be performed by a narrow band level set method or a pattern classification method based on maximum a posteriori (MAP) probability framework.

In step 205, first lesions may be identified in the segmented first anatomical image. The first lesions may comprise white matter lesions 213. The identification of the first lesions may for example be performed by comparing the segmented first anatomical image with a reference image e.g. that has no lesions of the same subject 118 and the same examination area. The differences between the two images may indicate the first lesions. Other techniques for identifying the lesions may be used. These techniques, may a) use spatial prior information e.g. in form of an atlas generated from a database of patients, b) analyze the gray value distribution in local areas around suspected lesions, comparing those actual distribution to the distribution in unaffected regions, and c) perform some post-processing, e.g. a connectivity analysis, removing lesions which are too small.

For example, to each identified lesion a unique ID and a label corresponding to its anatomical region may be assigned, where the anatomical region is identified by the result of the (automatic) segmentation of step 203.

In one example, steps 203 and 205 may be performed on respective different first anatomical images of the examination area. For example, step 203 may segment image 1 and step 205 may use image 2. In this case, the two images 1 and 2 have to be registered before performing step 205. For that the two images 1 and 2 (e.g. in step 203) may be segmented e.g. using the technique of shape-constrained deformable models, resulting in a mesh representation of the surface of anatomical structures in the two images. Then, based on the mesh vertices of the structures which are contained in both images, a (e.g. rigid or affine) transformation can be calculated registering the segmented mesh of one image to the segmented mesh of the other image. This transformation can then be applied to register the one image to the other image. This mesh registration may be used in other examples e.g. when having first anatomical images at two time points and have to be registered or when performing multi-modal segmentation with more than one anatomical modality, e.g. T1 and T2 or FLAIR.

In step 207, the identified first lesions may be used as seed points for a tracking algorithm for tracking first fibers in the first image of fibers. For example, a center of gravity of each lesion of the identified first lesions may be calculated. The resulting center of gravities may be used as the seed point for each lesion. In another example, a voxel having the highest or lowest intensity (depending on the imaging modality) in each lesion may be used as seed point for each lesion. In one example, step 207 may for example be performed using values of a first parameter and a second parameter that describe characteristics of the first anatomical image and the first image of fibers respectively. For example, the first anatomical image and the first image of fibers may automatically be scanned at the same time or concurrently in order to place a seed point in a given first lesion and to perform a comparison between the characteristics of the first anatomical image and the first image of fibers where the seed point is first placed in the given first lesion of the identified first lesions. Based on the comparison, the placed seed point may or may not be used for the tracking of fibers.

Consider for example, a given seed point within a candidate area (e.g. one of the identified first lesions of the first anatomical image). The given seed point may cover one or more voxels e.g. a voxel Vx. The second parameter may be evaluated for a corresponding voxel of Vx in the first image of fibers or may be evaluated for a region surrounding the corresponding voxel of Vx (also referred to as Vx) in the first image of fibers. The first image of fibers may for example be obtained using a diffusion tensor imaging method. The second parameter may for example comprise the direction of the diffusion, mean diffusivity, apparent diffusion coefficient, eigenvalues of the tensor in the voxel Vx in the first image of fibers etc. If for example the mean diffusivity of the voxel Vx in the first image of fibers is higher than a predefined threshold (e.g. a fastest diffusion would indicate the overall orientation of the fibers), the given seed point is accepted, and the given seed point may be used as input for the tracking algorithm to track the fibers starting from the given seed point. In another example, the set of eigenvalues of the diffusion tensor for voxel Vx in the first image of fibers is mapped by a potentially non-linear function to the real axis, and the given seed point may be accepted if the resulting value is above a predefined threshold value.

The tracking algorithm may comprise for example DTI Tractography or FiberTrak that enables to visualize white matter fibers in the brain and can map subtle changes in the white matter associated with diseases such as multiple sclerosis and epilepsy, as well as assessing diseases where the brain's wiring is abnormal, such as schizophrenia.

For example, the tracking may be performed in a region of interest of the first anatomical image. The region of interest may be user-defined or automatically selected. The automatic selection may for example be performed using the IDs and labels assigned to the identified first lesions.

For example, the user or the automatic selection may require access to all white matter lesions in basal ganglia e.g. the region of interest may comprise the basal ganglia.

In another example, the tracking may be performed in the whole region of the first anatomical image.

In one example, step 207 may further comprise displaying the tracked fibers and/or lesions in a graphical user interface as for example shown with reference to FIG. 5.

The lesion detection application 119 may comprise instructions that when executed perform automatically steps 201-207.

FIG. 3 is a flowchart of an exemplary method for performing a longitudinal analysis. Steps 201-207 of FIG. 2 may be repeated using a second anatomical image of the same examination area and a second image of fibers of the same examination area of the same subject. This may result in identified second lesions and tracked second fibers and a second affected cortical area in case the examination area comprises the brain.

In step 301, the first and second lesions may be compared and the first tracked fibers and the second tracked fibers. In case the examination area comprises the brain, step 301 may further comprise comparing the affected first and second cortical areas. Step 301 may for example be accomplished by calculating a difference image, i.e. subtracting voxel intensities of the second fiber image from the voxel intensities of the (registered and correspondingly normalized) first fiber image. Furthermore, statistical indices (e.g. total volume of affected fibers) together with their difference can be computed and displayed.

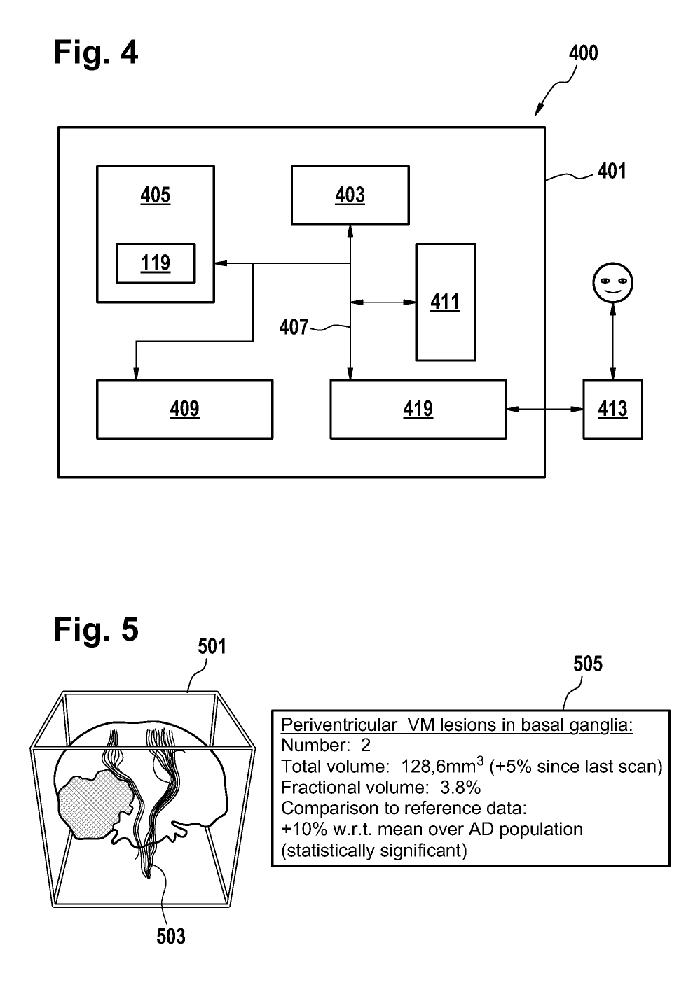

In step 303, data indicative of the difference between imaged first and second lesions may be provided and/or between the first and second tracked fibers. For example, that difference may be displayed on the graphical user interface. For example, the total volume variation between the current iteration and the previous one may be displayed as shown with reference to FIG. 4. In case the examination area comprises the brain, step 303 may further comprise displaying the affected first and second cortical areas. The displaying of the first and second affected cortical areas may be performed in a semi-transparent display mode while the intersection between the first and second affected cortical areas may be displayed in a non-transparent display mode. This may help tracking changes in the affected cortical areas.

Steps 201-303 may be repeated until a predefined convergence criterion is fulfilled (inquiry 305). For example, the display of the difference may further prompt the user to select a “continue” or a “stop” button on the graphical user interface. The selection of the “continue” button may trigger the repetition of steps 201-303. In another example, the repetition may be automatically triggered after a predefined display time interval e.g. if the user does not react (e.g. select one of the “continue” and “stop” buttons) in that predefined display time interval the method may be repeated. For each iteration or repetition a respective anatomical image and image of fibers of the same examination area of the same patient or subject may be used. Each iteration or repetition may result in a respective identified lesion and tracked fibers.

The convergence criterion may comprise receiving a stopping signal upon performing step 303. For example, the user may select the “stop” button. In another example, the repetition may be stopped if the difference between the imaged lesions of the current iteration and image lesions of the immediately preceding iteration is below a predefined threshold. The stopping of the repetition may be performed automatically by comparing the difference with the predefined threshold.

In a further example, in case the number of second lesions is equal to the first number of lesions, the repetition of steps 201-203 may be stopped.

FIG. 4 depicts a functional block diagram illustrating a medical instrument 400 in accordance with the present disclosure.

The medical instrument 400 may comprise an image processing system 401. The components of image processing system 401 may include, but are not limited to, one or more processors or processing units 403, a storage system 411, a memory unit 405, and a bus 407 that couples various system components including memory unit 405 to processor 403. Storage system 411 may include a hard disk drive (HDD). Memory unit 405 may include computer system readable media in the form of volatile memory, such as random access memory (RAM) and/or cache memory.

Image processing system 401 typically includes a variety of computer system readable media. Such media may be any available media that is accessible by image processing system 401, and it includes both volatile and non-volatile media, removable and non-removable media.

Image processing system 401 may also communicate with one or more external devices such as a keyboard, a pointing device, a display 413, etc.; one or more devices that enable a user to interact with image processing system 401; and/or any devices (e.g., network card, modem, etc.) that enable image processing system 401 to communicate with one or more other computing devices. Such communication can occur via I/O interface(s) 419. Still yet, image processing system 401 can communicate with one or more networks such as a local area network (LAN), a general wide area network (WAN), and/or a public network (e.g., the Internet) via network adapter 409. As depicted, network adapter 409 communicates with the other components of image processing system 401 via bus 407.

Memory unit 405 is configured to store applications that are executable on the processor 403. For example, the memory system 405 may comprise an operating system as well as application programs. The application programs comprise for example the lesion detection application 419. The lesion detection application 119 comprises instructions that when executed lesion detection application 119 may receive as inputs or may access existing two images to be processed in accordance with the present disclosure (e.g. as described with reference to FIG. 2 and FIG. 3). The execution of the instructions may further cause the processor 403 to display a graphical user interface on the display 413.

FIG. 5 depicts a schematic visualization of white matter fibers 503 affected by white matter lesions in a user-defined anatomical area 501 and a display of the results 505 of a statistical analysis of the selected white matter lesions.

The statistical analysis may be carried out on the identified white matter lesions (e.g. in the region of interest), and the white matter fibers which are affected by the white matter lesions are extracted. The results are visualized in a convenient format. For example, the selected white matter lesions can be overlaid on the affected fiber tracts. In addition, the patients' anatomy can be overlaid in a semi-transparent way. Alternatively, the surface of the selected area of interest (extracted from the automatic segmentation algorithm) can be overlaid in a semi-transparent way. The statistical assessment of the white matter lesions in the selected region of interest may comprise e.g. the number of white matter lesions, their total volume, their fractional volume (total volume of white matter lesions divided by total volume of the region), comparison of the statistical indices to reference databases and/or to a previous scan of the patient etc.). The results of the statistical assessment are visualized in a convenient way in a graphical or textual form (505). As an example for a graphical representation, fractional volumes could be visualized in form of “heat maps”, total volumes as bar charts etc.

In the following another exemplary method for identifying white matter lesions and affected fibers is described. This method may have the advantage of handling in an efficient manner all white matter lesions in an anatomical region of interest. This method may provide an automatic regional or global analysis of statistical indices of white matter lesions, like size, number, scores, fractional volumes, percentage of deviation to reference database or previous scan etc. (“regional” refers to anatomical regions of interest like basal ganglia). Also, the visualization of a single (or all) white matter lesions together with the associated (affected) fibers and the overall anatomy is provided in a convenient and efficient way of selecting anatomical regions of interest for white matter lesion assessment and visualization of affected fibers.

This method may consist of an automated seed point placement in white matter lesions in an anatomical region of interest (e.g. from MR T1 image) for automatic fiber tracking in a co-registered MR DTI image. The present method may further comprise a selection and visualization of the white matter lesions contained in the user-selected region of interest; visualization of the corresponding (i.e. affected) white matter tracts and visualization of the underlying anatomy (semi-transparent). Additionally or alternatively, visualization of the surface of the selected (sub-cortical) area may be provided. The present method may further comprise an automatic generation of a region-wise white matter lesion profile, e.g. determining the size, number, fractional volume (volume of white matter lesions within the selected region divided by the volume of the region), percentage of deviation to reference database or previous scan etc.; visualization/display of the results in a convenient user interface in various forms (e.g. in textual or graphical form) to be customized by the user.

The method may comprise the following steps:
An automatic segmentation algorithm comprising the relevant anatomical structures and regions may be applied to an anatomical image, e.g. a MR T1 image e.g. of the brain of a patient.
Automatically annotating the white matter lesions using a selected conventional algorithm. To each annotated white matter lesion, a unique ID and a label corresponding to its anatomical region can be assigned, where the anatomical region is identified by the result of the automatic segmentation (if white matter lesions and automatic segmentation are determined in different images, the two images have to be registered using state-of-the-art registration algorithms).
For each annotated white matter lesion (identified e.g. by a connected component analysis), the center-of-gravity is calculated (alternatively, e.g. for extended white matter lesions, a dense set of points covering the extent of the white matter lesion may be determined). These points are consecutively used as seed points for a fiber tracking algorithm applied to the MR DTI image which are registered to the anatomical image using a registration algorithm. In this way, the white matter tracts passing through each individual white matter lesion are automatically being determined. Furthermore, a label is assigned to the determined white matter tracts indicating the anatomical region of the corresponding white matter lesion.
The user can then select an anatomical region of interest—which may be supported by the segmentation algorithm—in a convenient user interface (the graphical user interface described above). For example, the user may select individual sub-cortical structures of interest (e.g. globus pallidus) or regions (e.g. basal ganglia).
The selected region is then used to filter out the white matter lesions which are contained in this specific region (i.e. which have the corresponding anatomical label). Then, a statistical analysis is carried out on the subset of the white matter lesions, and the white matter fibers which are affected by the selected white matter lesions are extracted (via the associated anatomical label). The results are then visualized in a convenient format, see FIG. 5. For example, the selected white matter lesions can be overlaid on the affected fiber tracts. In addition, the patients' anatomy can be overlaid in a semi-transparent way. Alternatively, the surface of the selected area of interest (extracted from the automatic segmentation algorithm) can be overlaid in a semi-transparent way.
The statistical assessment of the white matter lesions in the selected region of interest may comprise e.g. the number of white matter lesions, their total volume, their fractional volume (total volume of white matter lesions divided by total volume of the region), comparison of the statistical indices to reference databases and/or to a previous scan of the patient etc.). The results of the statistical assessment are visualized in a convenient way in a graphical or textual form (505). As an example for a graphical representation, fractional volumes could be visualized in form of “heat maps”, total volumes as bar charts etc.

### LIST OF REFERENCE NUMERALS

100 magnetic resonance imaging system
104 magnet
106 bore of magnet
108 imaging zone
110 magnetic field gradient coils
112 magnetic field gradient coil power supply
114 radio-frequency coil
115 RF amplifier
118 subject
119 lesion detection application
126 computer system
128 hardware interface
130 processor
132 user interface
134 computer storage
136 computer memory
160 control module
201-207 steps
209 anatomical image
211 segments
213 white matter lesions
400 medical instrument
401 image processing system
403 processor
405 memory
407 bus
409 network adapter
411 storage system
413 display
419 I/O interface
501 user-defined anatomical area
503 fibers
505 display results.

1. A medical instrument for automatically detecting affected regions in an examination area of a subject, comprising: a memory containing machine executable instructions; and a processor for controlling the medical instrument, wherein execution of the machine executable instructions causes the processor to control the instrument to:
a) obtain a first anatomical image of the examination area and a first image of fibers of the examination area, wherein a first parameter and a second parameter describe characteristics of the first anatomical image and the first image of fibers respectively;
b) segment the first anatomical image into a plurality of segments indicating respective tissues and/or structures in the examination area;
c) identify first lesions in the segmented first anatomical image;
d) use values of the first and second parameters for determining seed points in the identified first lesions for a tracking algorithm for tracking first fibers (503) in the first image of fibers.

2. The medical instrument of claim 1, wherein execution of the machine executable instructions further causes the processor to control the instrument to:
e) obtain a second anatomical image of the examination area and a second image of fibers of the examination area;
f) segment the second anatomical image into a plurality of segments indicating respective tissues and/or structures in the examination area;
g) identify second lesions in the segmented second MR image;
h) use the identified second lesions as seed points for the tracking algorithm for tracking second fibers in the second image of fibers;
i) compare at least the first and second lesions;
j) provide data indicative of the difference between imaged first and second lesions and
repeat steps e)-j) until a predefined convergence criterion is fulfilled.

3. The medical instrument of claim 2, wherein the convergence criterion comprises at least one of:
the difference between the imaged first and second lesions is below a predefined threshold;
receiving a stopping signal upon performing step j);
the number of second lesions is equal to the number of the first lesions.

4. The medical instrument of claim 1, wherein execution of the machine executable instructions causes the processor to control the instrument to perform the tracking in a region of interest of the first anatomical image.

5. The medical instrument of claim 4, wherein the region of interest is automatically selected.

6. The medical instrument of claim 1, wherein the first anatomical image comprises a magnetic resonance, MR, image and the first image of fibers comprises a diffusion weighted image.

7. The medical instrument of claim 6, further comprising a magnetic resonance imaging, MRI, system for acquiring magnetic resonance data from the subject, wherein the magnetic resonance imaging system comprises a main magnet for generating a BO magnetic field within an imaging zone and the memory and the processor, wherein execution of the machine executable instructions further causes the processor to control the MRI system to acquire the MR image and the diffusion-weighted image in a same or different scans.

8. The medical instrument of claim 7, wherein execution of the machine executable instructions further causes the processor to acquire the MR image and the diffusion-weighted image in different scans and to register the MR image and the diffusion-weighted image before performing steps a)-d).

9. The medical instrument of claim 1, wherein execution of the machine executable instructions further causes the processor to calculate a center of gravity of each lesion of the lesions and use the center of gravities as the seed points.

10. The medical system of claim 1, wherein the first parameter comprises at least one of size, number, voxel intensity, fractional volume of the identified lesions, wherein the second parameter comprises at least one of direction of diffusion and the magnitude of the diffusion.

11. The medical instrument of claim 2, wherein the provided data comprises characteristics of the lesions such as size, number, fractional volume of the lesions.

12. The medical instrument of claim 1, wherein the first lesions comprises white matter lesions, and the examination area comprises a brain.

13. A computer program product for automatically detecting affected regions in an examination area of a subject, the computer program product comprising a computer readable storage medium having program instructions embodied therewith, the program instructions executable by a processor to
a) obtain a first anatomical image of the examination area and a first image of fibers of the examination area, wherein a first parameter and a second parameter describe characteristics of the first anatomical image and the first image of fibers respectively;
b) segment the first anatomical image into a plurality of segments indicating respective tissues and/or structures in the examination area;
c) identify first lesions in the segmented first anatomical image;
d) use values of the first and second parameters for determining seed points in the identified first lesions for a tracking algorithm for tracking first fibers in the first image of fibers.

14. A method comprising:
a) obtaining a first anatomical image of an examination area of a subject and a first image of fibers of the examination area, wherein a first parameter and a second parameter describe characteristics of the first anatomical image and the first image of fibers respectively;
b) segmenting the first anatomical image into a plurality of segments indicating respective tissues and/or structures in the examination area;
c) identifying first lesions in the segmented first anatomical image;
d) using values of the first and second parameters for determining seed points in the identified first lesions for a tracking algorithm for tracking first fibers in the first image of fibers.
